(19) United States
(12) Patent Application Publication
(10) Pub. No.: US 2006/0292695 A1
(43) Pub. Date: Dec. 28, 2006

(54) Methods and kits for drug screening and toxicity testing using promoter-reporter cells derived from embryonic stem cells

(75) Inventors: A. John Clark, Midlothian (GB); Helen Clark, Midlothian (GB); C. Roland Wolf, Dundee (GB)

Correspondence Address: GERON CORPORATION, 230 CONSTITUTION DRIVE, MENLO PARK, CA, 94025, US

(73) Assignees: Roslin Institute, Midlothian (GB); CXR Biosciences Ltd., Dundee (GB)

(21) Appl. No.: 11/233,819

(22) Filed: Sep. 22, 2005

Related U.S. Application Data
(60) Provisional application No. 60/693,319, filed on Jun. 22, 2005.

(51) Int. Cl. C12N 5/08

(52) U.S. Cl. 435/370; 435/4

(57) ABSTRACT

This invention provides a system for rapid determination of pharmacologic effects on target tissue types in cell populations cultured in vitro. The cells contain a promoter-reporter construct that reflects a toxicologic or metabolic change caused by the agent being screened. The promoter is taken from a gene known to be up- or down-regulated according to the metabolic state of the cell, and linked to a reporter gene that provides an external signal for monitoring promoter activity. The promoter-reporter cells may be produced by placing these genetic alterations into a line of human embryonic stem cells, bulking up the cells to any extent desired, and then differentiating the cells into the desired tissue type. This disclosure explains some of the powerful features of the promoter-reporter cells of this invention, and shows various ways the skilled reader can use the invention for pharmaceutical development and testing, or to monitor graft survival.

### REFERENCE TO PREVIOUS APPLICATION

This application claims the priority benefit of U.S. Provisional Patent Application 60/693,319 (Docket 140/001x), filed Jun. 22, 2005. The priority document is hereby incorporated herein by reference in its entirety.

### BACKGROUND

A key unmet need in pharmaceutical development is reliably available, cost-effective and predictive models for determining the metabolic and toxicological properties of drug compounds. Current in vitro models such as primary hepatocytes suffer from inconsistent availability and significant phenotypic variability. In vivo animal models are prohibitively expensive, have low throughput, and are often not predictive for humans.

As a result, a compound's metabolic and toxicological properties are often not studied until late in preclinical development, requiring pharmaceutical companies to invest significant resources in a compound's development in the absence of information about these most critical traits. Unfortunately, the results obtained in late preclinical animal studies often fail to predict problems subsequently seen in early human trials, resulting in high failure rates and risk to volunteers in Phase I trials.

Human embryonic stem (hES) cells (U.S. Pat. No. 6,200,806) present a unique opportunity to address this need. Undifferentiated hESCs have a virtually indefinite replicative capacity in culture. Recent developments have enabled the economically viable production of commercial quantities of undifferentiated cells. See, for example, U.S. Pat. No. 6,800,480; WO 01/51616; WO 03/020920; Rosier et al., Dev Dyn. 2004; 229(2):259-74; Xu et al., Stem Cells 2005; 23(3):315-23; and Li et al., “Expansion of human embryonic stem cells”, Biotechnology and Bioengineering, Published Online: 21 Jun. 2005.

Geron Corporation has previously shown how hES cells can be directed to differentiate into cells of a particular phenotype en masse, generating high quality cell populations with reproducible standards. For example, U.S. Pat. Nos. 6,458,589 and 6,506,574; WO 01/81549; Rambhatla et al., Cell Transplant. 2003; 12(1):1-11; and US 2005/0037493 A1 describe procedures for generating hepatocyte lineage cells. hES cells can be used to generate other cell types that are of particular interest to the drug screening industry.

This disclosure capitalizes on some of the unique properties of hES cells and their derivatives, providing genetically modified cells that provide rapid readout of pharmacologic and toxicologic effects.

### SUMMARY

This disclosure provides a new system for drug and environmental screening and analysis, using cells that report changes in the culture environment—such as the presence of a drug being screened for its effects on the same tissue type in vivo.

One aspect of the invention is undifferentiated human embryonic stem (hES) cells, hepatocyte-like cells, or hES derived cell products containing a promoter-reporter system. The cells have been genetically altered so that a promoter that responds to a metabolic or toxicologic change in the cell controls expression of a reporter gene. Suitable classes of promoters and reporter genes, along with examples of each are given in the sections that follow.

Exemplary differentiated cell types represent the neural, cardiomyocyte, or hepatocyte lineages, having characteristic markers of such cells. The user may determine that a particular cell population is derived by hES cells by examining the cell population as a whole. Characteristics of other cell types in the preparation will distinguish hES derived cells from cells obtained from other sources. Differentiated cells of this invention may also contain a second promoter-reporter system to identify a particular cell type within a mixed population.

Another aspect of the invention is a method for producing a differentiated cell population with a promoter-reporter system, by genetically altering undifferentiated hES cells so that a promoter that responds to a metabolic or toxicologic change in the cell controls expression of a reporter gene. The cells are then proliferated and differentiated into a particular cell type of interest to the user.

Another aspect of the invention is a method for drug testing, in which the drug is combined with a promoter-reporter cell population of this invention, and the user determines whether there is a change in expression of the reporter gene. Expression may be measured in the cell population as a whole, or in a particular cell marked by a second reporter gene under control of a tissue specific promoter. Drug targets or enzymes believed to be involved in metabolism of the drug can be validated using RNAi.

Another aspect of this invention is a kit or combination of reagents, comprising the promoter-reporter cells of this invention, optionally in combination with one or more other cell populations derived from the same hES cell line and sharing the same genome. The matched cell populations may contain cells having a different promoter-reporter system, cells differentiated into another cell type, or cells having an allelic variant of a drug target or drug metabolizing enzyme.

Another aspect of this invention is a system for monitoring graft survival. Isolated tissue is adapted for transplantation by genetically altering at least some of the cells in the tissue with a promoter-reporter construct. The promoter may respond to oxidative stress or apoptosis; it may reflect cell viability or graft rejection. The reporter can encode a product secreted from the cell and possibly excretable by the kidney, enabling status of the graft to be followed in the blood or urine.

These and other embodiments of the invention are described in the sections that follow.

### DRAWINGS

FIG. 1 illustrates a protocol for making hepatocyte lineage cells from hES cells, exemplified in Example 1 of this disclosure. Upper Panel shows the differentiation scheme. Differentiation is initiated using DMSO, and proceeds using a combination of growth factors (epidermal growth factor, EGF; hepatocyte growth factor, HGF), a glucocorticoid (dexamethasone, Dex), and Oncostatin M (OSM). The culture was further matured by culturing with HGF. Middle Panel shows the resultant cells having morphological features of hepatocytes. Bottom Panel shows expression of various cell markers as detected by RT-PCR (real-time PCR amplification of mRNA), through the various stages of the differentiation protocol.

FIG. 2 maps the human α-fetoprotein (AFP) and CYP3A4 promoters used for model promoter-reporter constructs of this invention. Upper Panel: AFP Transcription regulatory region (5.4 kb) contains E_{A}: enhancer domain A; E_{B}: enhancer domain B; Sd: distal silencer; Sp: proximal silencer; P: promoter. Lower Panel: CYP3A4 regulatory region contains a 0.8 kBase xenobiotic-responsive enhancer module (XREM) and a 0.4 kBase mini proximal promoter (P). Total size is about 1.2 kb.

FIG. 3 shows the staining patterns during differentiation of hES cells to hepatocyte lineage cells. Panels at Top of Page: Row A is the phase contrast image; Row B shows immunostaining for hepatocyte markers; Row C shows uptake and clearance of indocyanine green. hESCs were cultured first in conditioned medium (Left Column), then in DMSO medium for 7 days to endoderm-like morphology (Middle Column); finally in hepatocyte differentiation medium for ˜2 weeks, forming foci of hepatocyte lineage cells (HLCs) (Right Column). Panels at Bottom of Page show expression of hepatocyte markers measured by RT-PCR. Results are shown for the G4 Cell line (Left Panel) and the H1Parental line (Right Panel). Abbreviations used: ES: hES cells; HLC: isolated foci of hES derived hepatocyte lineage cells; F: fetal hepatocytes; A: adult hepatocytes.

FIG. 4 shows expression of the reporter GFP in H1-LZ-hAFP-eGFP clonal cell lines after differentiation. Row A shows expression of AFP-GFP transgene in HLCs, visualized by GFP staining (Left), phase contrast (Middle), or overlay (Right). Row B shows colocalization of AFP-GFP with endogenous AFP protein in HLCs, as shown by GFP staining (Left), AFP staining (Middle), or overlay (Right). Row C shows that-GFP is not expressed in the primitive endoderm of embryoid bodies, though endogenous AFP is present, as shown by GFP staining (Left), AFP staining (Middle), or phase contrast (Right).

FIG. 5 shows immunostaining of HLCs in H1-LZ-hAFP-eGFP clonal cell lines. The H1-LZ-hAFP-eGFP hESCs were differentiated to HLCs and stained with antibodies against GFP (representing expression of AFP-GFP transgene) and hepatocyte markers. Row A: albumin staining; Row B: HepPar1 staining; Row C: PXR staining; Row D: aAT staining. First Column: GFP staining; Second Column: marker staining; Third Column: co-localization.

FIG. 6 shows hES derived hepatocytes in which expression of the GFP reporter is controlled by the promoter for the cytochrome P450 enzyme CYP3A4-GFP. Top Row shows the phase contrast image. Bottom Row shows expression of the reporter gene as detected by fluorescence. Left Column (−) shows endogenous expression; Right Column (+) shows expression induced upon treatment with rifampicin and dexamethasone.

FIG. 7 shows metabolism of compounds by hES cell derived promoter-reporter hepatocytes. Top Panel: midazolam; Bottom Panel: tolbutamide, which are processed by the CYP3A4 and CYP2C9 isozymes of cytochrome P450, respectively. These results demonstrate that the hESC derived cells have inducible cytochrome P450 activity characteristic of pharmacologically active hepatocytes, and so have appropriate activity for use in drug screening assays.

### DETAILED DESCRIPTION

This invention provides a system for rapid determination of pharmacologic effects on target tissue types in cell populations cultured in vitro. The cells contain a promoter-reporter construct that reflects a toxicologic or metabolic change in the cell, such as may be caused by a drug present in the culture medium. The promoter is taken from a gene known to be upregulated when a particular toxicologic or other metabolic effect takes place in the cell. It controls transcription of a reporter gene that provides an external signal that can be monitored as an indication of promoter activity. This system enables rapid high-throughput screening of a panel of test agents for potential toxicity and other effects on the cell.

Many aspects of this invention capitalize on the special properties of human embryonic stem cells. In particular:
hES cells can provide a virtually limitless supply of differentiated cells having the same genome and a reproducible phenotype—ensuring consistency of production and reproducible screening assays.
Using hES cells as the source allows promoter-reporter systems to be built into non-cancer-derived cell types that are otherwise not amenable to genetic modification due to limited replicative capacity—such as hepatocytes.
The ability to make multiple modifications to the same cell line enables the development of sophisticated double-label systems, whereby multiple metabolic effects can be followed simultaneously—on a cell-by-cell basis in a mixed population, if necessary.
The ability to direct hES cells into different cell lineages enables the development of matched cell populations where the effects of compounds on different tissues can all be measured in vitro.
hES cells can be used to create different lines that are genetically identical, except for variations in important drug metabolizing enzymes, such as CYP2D6; or drug targets such as G protein coupled receptors (GPCR)—enabling the user to determine drug effects that are variant-dependent.
   The description and examples that follow show some of the powerful features of the promoter-reporter cells of this invention, and explain how the reader can use them for a variety of purposes related to pharmaceutical development and testing, and in human clinical therapy.
   Definitions

Prototype “primate Pluripotent Stem cells” (pPS cells) are pluripotent cells derived from pre-embryonic, embryonic, or fetal tissue at any time after fertilization, that have the characteristic of being capable under appropriate conditions of producing progeny representing each of the three germ layers: endoderm, mesoderm, and ectoderm, as determined by a standard art-accepted test, such as the ability to form a teratoma in a suitable host, or the ability to differentiate into cells stainable for markers representing tissue types of all three germ layers in culture.

Prototype “human Embryonic Stem cells” (hES cells) are described by Thomson et al. (Science 282:1145, 1998; U.S. Pat. No. 6,200,806). The scope of the term covers pluripotent stem cells that are derived from a human embryo at the blastocyst stage, or before substantial differentiation of the cells into the three germ layers. Except where explicitly required otherwise, the term includes primary tissue and established lines that bear phenotypic characteristics of hES cells, and progeny of such lines that still have the capacity of producing progeny of each of the three germ layers. Reference in this description to the manipulation and use of hES cells and their derivatives will be understood to be applicable to other pPS cells mutatis mutandis, unless otherwise prohibited.

pPS and hES cell cultures are described as “undifferentiated” when a substantial proportion of stem cells and their derivatives in the population display morphological characteristics of undifferentiated cells, and maintain their ability to differentiate into all three germ layers. Colonies of undifferentiated cells will often be surrounded by neighboring cells that are differentiated. Nevertheless, the undifferentiated colonies persist when cultured or passaged under appropriate conditions, such that undifferentiated cells constitute a substantial proportion of the cell population. “Differentiated” cells have been cultured or maintained in such a manner so that the cells lose the ability to differentiate into all three germ layers, typically accompanied by a morphological change. Exemplary differentiated cells have characteristics of a particular tissue type, such as hepatocytes, cardiomyocytes, nerve cells, islet cells, and hematopoietic cells.

A “promoter” is a DNA sequence involved in initiating transcription of the encoding region of a gene to which it is linked. It may cause constitutive expression of the gene, it may be upregulated in a tissue-specific way, or it may be upregulated in response to a metabolic or toxicologic effect. Embodiments of this invention using promoters with particular specificity are equivalent mutatis mutandis to combinations of other transcriptional control elements having the same specificity—such as an enhancer that controls the specificity of a promoter to which it is linked.

A “reporter gene” is any nucleic acid sequence which, when expressed in a cell, causes the cell to display a detectable label, such as a fluorescent or phosphorescent signal, an enzyme activity detectable in an assay, or an antigen detectable on or in the cell by a specific stain, antibody, or lectin.

Genetic elements are “operatively linked” if they are in a structural relationship permitting them to operate in a manner according to their expected function. A promoter is operatively linked to an encoding region if the promoter drives transcription of the encoding region. There may be an intervening sequence between the promoter and encoding region so long as this functional relationship is maintained.

A cell is said to be “genetically altered”, or “transfected” when a polynucleotide has been transferred into the cell by any suitable means of artificial manipulation, or where the cell is a progeny of the originally altered cell that has inherited the polynucleotide. A genetic alteration is said to be “stable” if it is inheritable through at least 4 passages of cell culture, detectable as the presence of the polynucleotide template in a 7^{th} generation cell. Alterations to the cell genome (insertion of a transgene or inactivation of an endogenous gene) are usually stable. A genetic alteration is said to be “transient” if it dilutes away upon replication or extended culturing of the cell. This includes transient transfection with adenovirus vectors or plasmids.

An “expression system” is a control element operatively linked to a coding region so that the control element drives expression of the coding region.

A “promoter-reporter construct” is a recombinant polynucleotide inside or outside a cell, in which a promoter is operatively linked to a reporter gene, A “promoter reporter cell” is a cell genetically altered (either stably or transiently) so as to contain a promoter reporter construct, wherein compounds that activate the promoter cause expression of the reporter gene in the cell.

A “cell line” is a population of cells that can be propagated in culture through at least 10 passages without substantial change in phenotype. The population can be phenotypically homogeneous, or the population can be a mixture of measurably different phenotypes. Characteristics of the cell line are those characteristics of the population as a whole that are essentially unaltered after 10 passages.

Cell lines of this invention are typically “non-cancer-derived”. This means that they have not been derived from a cancer cell or transduced with an oncogene, and lack the genetic and phenotypic features characteristic of cancer-derived cells.

The term “drug target” as used in this disclosure refers to a biological molecule or biochemical pathway in a cell or tissue that mediates a pharmacological effect (such as an intended therapeutic effect or a side-effect) of a particular drug of interest. The pharmaceutical effect may but does not necessarily result from direct binding of the drug to the drug target.

The term “drug metabolizing enzyme” as used in this disclosure means any protein or nucleoprotein on or attached to a cell that chemically alters or sequesters a drug or class of drugs. Unless explicitly indicated otherwise, the term includes chemical disassembly, conjugation, or conversion to another compound, or transport to another intercellular or intracellular space, as long as this results in elimination of the drug or substantial modification of its effect.

General References

General methods in cell biology, protein chemistry, and antibody techniques can be found in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Reagents, cloning vectors, and kits for genetic manipulation referred to in this disclosure are available from commercial vendors such as BioRad, Stratagene, Invitrogen, and ClonTech.

Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Tissue culture supplies and reagents are available from commercial vendors such as Gibco/BRL, Nalgene-Nunc International, Sigma Chemical Co., and ICN Biomedicals.

Specialized reference books of interest include The Hepatocyte Review, M. N. Berry & A. M. Edwards Eds., Kluwer Academic Publishers, 2000; Stem Cell and Liver Regeneration, Kiwamu Okita, Springer-Verlag 2004; and The Reporter's Handbook, S. Weinberg et al., St. Martin's Press 1995. References on drug screening and assessment include Handbook of Drug Screening, R. Seethala & P. B. Femandes Eds., Marcel Dekker, 2001; Bioassay Techniques for Drug Development, Atta-Ur-Rahman et al., Taylor & Francis, 2001; and Cytochrome P450: Structure, Mechanism, and Biochemistry, P. R. Ortiz de Montellano, Kluwer Academic/Plenum, 2005.

Sources of Stem Cells

This invention can be practiced using stem cells of various types. Suitable for use in many aspects of the invention are pluripotent stem cell derived from any human or other primate tissue that meet the required definition. Non-limiting examples are primary cultures or established lines of human embryonic stem (hES) cells.

Embryonic Stem Cells

Embryonic stem cells can be isolated from blastocysts of primate species (U.S. Pat. No. 5,843,780; Thomson et al., Proc. Natl. Acad. Sci. USA 92:7844, 1995). hES cells can be prepared from human blastocysts using the techniques described by Thomson et al. (U.S. Pat. No. 6,200,806; Science 282:1145, 1998; Curr. Top. Dev. Biol. 38:133, 1998); Reubinoff et al, Nature Biotech. 18:399, 2000; and Genbacev et al., Fertil Steril. 2005; 83(5):1517-29. Equivalent cell types to hES cells include their pluripotent derivatives, such as primitive ectoderm-like (EPL) cells, outlined in WO 01/51610 (Bresagen). Also equivalent are cells that have been reprogrammed into pluripotent stem cells by fusion with hES cells.

hES cells can be propagated using culture conditions that promote proliferation while inhibiting differentiation. Traditionally, hES cells are cultured on a layer of feeder cells, typically fibroblasts derived from embryonic or fetal tissue (Thomson et al., Science 282:1145, 1998).

hES cells can also be maintained in an undifferentiated state without feeder cells in a specially designed culture environments Feeder-free cultures often includes an extracellular matrix, such as Matrigel® or laminin. The cultures are supported by a nutrient medium containing factors that promote proliferation of the cells in the undifferentiated form (WO 99/20741). Such factors may be introduced into the medium by culturing the medium with cells secreting such factors, such as irradiated primary mouse embryonic fibroblasts, telomerized mouse fibroblasts, or fibroblast-like cells derived from hES cells (U.S. Pat. No. 6,642,048; U.S. Pat. No. 6,800,480; WO 01/51616; Xu et al., Nat. Biotechnol. 19:971, 2001).

Alternatively, fresh non-conditioned medium can be used, if supplemented with directly added factors (like a fibroblast growth factor or forskolin) that promote proliferation of the cells in an undifferentiated form. Exemplary is a base medium like X-VIVO™ 10 (Biowhittaker) or QBSF™-60 (Quality Biological Inc.), supplemented with bFGF at 40-80 ng/mL, and optionally containing stem cell factor (15 ng/mL), or Flt3 ligand (75 ng/mL). These medium formulations have the advantage of supporting cell growth at 2-3 times the rate in other culture systems (WO 03/020920).

Under the microscope, ES cells appear with high nuclear/cytoplasmic ratios, prominent nucleoli, and compact colony formation with poorly discernable cell junctions. Primate ES cells typically express the stage-specific embryonic antigens (SSEA) 3 and 4, and markers detectable using antibodies designated Tra-1-60 and Tra-1-81. Undifferentiated hES cells also typically express the transcription factor Oct-3/4, Cripto, and human telomerase reverse transcriptase (hTERT), as detected by RT-PCR (US 2003/0224411 A1).

Other Stem Cells

The illustrations provided in the Example section ensue from work done with hES cells. However, except where otherwise required, the invention can be practiced using multipotent cells of any vertebrate species, including pluripotent stem cells from humans, non-human primates, and other non-human mammals.

By no means does the practice of this invention require that a human blastocyst be disaggregated in order to produce the hES or embryonic stem cells for practice of this invention. hES cells can be obtained from established lines obtainable from public depositories (for example, the WiCell Research Institute, Madison Wis. U.S.A., or the American Type Culture Collection, Manassas Va., U.S.A.). Human Embryonic Germ (hEG) cells can be prepared from primordial germ cells as described in Shamblott et al., Proc. Natl. Acad. Sci. U.S.A. 95:13726, 1998 and U.S. Pat. No. 6,090,622. U.S. Patent Publication 2003/0113910 A1 reports pluripotent stem cells derived without the use of embryos or fetal tissue. It may also be possible to reprogram other progenitor cells into hES cells by using a factor that induces the pluripotent phenotype (Chambers et al., Cell 113:643, 2003; Mitsui et al., Cell 113:631, 2003). Under appropriate conditions, any cell with appropriate proliferative and differentiation capacities can be used for the derivation of differentiated tissues for use according to this invention.

The Promoter-Reporter System

The cells of this invention are designed to have one or more reporter genes expressed under control of a promoter or other transcription regulator sequence that responds to a drug or other aspect of the culture environment that affects the gene expression pattern in the cell. In some instances, the cells are engineered to have a second (tissue-specific) promoter that allows the user to identify a cell of interest to be identified amongst a mixed cell population.

Promoters that Respond to Metabolic or Toxicologic Changes

Any promoter or transcription control element controlling a gene that is up- or down-regulated in response to a change in culture conditions (particularly the presence of a class of test drugs) may be suitable for use in this invention. Examples of promoters having suitable characteristics include the following:
Promoters for genes that respond to apoptosis, such as the PUMA gene. Drugs that trigger apoptosis may trigger promoters in this category. Other candidates are Gadd34, PUMA, GAHSP40, TRAIL-R2/DR5, c-fos, Gaddl53, APAF-1, Gadd45, BTG2/PC3, Peg3/Pwl, Siahla, S29 ribosomal protein, FasUCD95L, tissue transglutaminase, GRP78, Nur77/NGFI-B, Cyclophilin D/CYPD, and P73.
Promoters for genes that respond to DNA damage, such as the p21, p21WAF1, or Pig3 gene. Mutagens or teratogens may trigger promoters in this category.
Promoters for genes that respond to hyperplasia, such as the Ki67 or Aurora A gene. Drugs that stimulate proliferation may trigger promoters in this category.
Promoters for genes that respond to oxidative stress. Heme oxygenase 1 (Hmox1), and superoxide dismutase (MnSOD) are upregulated with low oxygen levels; γ-glutamyl cysteinyl ligase (GCL), and Metallothionine I and II are upregulated by depletion of glutathione, or the presence of metal ions, respectively. Other candidates are IkB, ATF4, xanthine oxidase, COX2, iNOS, Ets-2, Cyclophilin A/CYPA, NQO1, and bNIP3.
Promoters for transcription factors that reflect changes in gene expression profiles upon initiation of any of these events, such as the PXR, CAR, aryl hydrocarbon receptor (AhR), or Nrf2 gene
Promoters for other hepatocyte markers that are upregulated in liver toxicity, such as Lrg-21, SOCS-2, SOCS-3, PAI-I, GBP28/adiponectin, α1-acid glycoprotein, ATF3, and lgfbp-3.
Promoters for genes that are responsive to receptors that act in the nucleus, exemplified by androgen, estrogen, and pPAG responsive gene. An example is the gene for prostate specific antigen (PSA).
Promoters for hepatocyte enzymes involved in drug metabolism that are also upregulated in the presence of substrate. Exemplary are cytochrome P450 genes, such as CYP3A4 and CYPLA1.
Promoter for drug transporter genes also upregulated by substrate, such as MDR1.
Promoters for genes that affect the contraction rate or the QT interval of the heart, such as calcium flux genes.
Promoters for genes controlling a product that is deficient in certain clinical conditions, and for which it may be useful to screen drugs that can regulate expression. Exemplary are genes that control hormone expression (e.g., insulin, or cortisol), and genes that control synthesis, release, metabolism, or reuptake of neurotransmitters (e.g., the serotonin transporter and tyrosine hydroxylase).
   These and other promoters referred to in this disclosure can be cloned by amplification from a suitable genomic library using primers specific for the desired sequence, constructed using sequence data from such sources as GenBank.
   Tissue Specific Promoters

To provide a countermarker to identify cells of a particular phenotype in the population, the user can select a second promoter that is constitutively expressed at some level, regardless of whether test compounds are present in the culture medium.

Cell markers specific for liver progenitors, hepatocytes, and biliary epithelium, are shown in Table 1.

TABLE 1
Liver Cell Markers

| | early | | biliary | | early | | biliary |
|---|---|---|---|---|---|---|---|
| | progenitors | hepatocytes | epithelium | | progenitors | hepatocytes | epithelium |
| albumin | + | + | − | OC.1 | − | − | + |
| α₁-antitrypsin | + | + | − | OC.2 | + | − | + |
| α-fetoprotein | + | fetal & | − | OC.3 | + | − | + |
| | | postnatal | | | | | |
| CEA | − | − | + (?) | BD.1 | + | − | + |
| γ-glutamyl | + | fetal | + | A6 | + | − | + |
| transpeptidase | | | | | | | |
| GST-P | + | fetal | + | HBD.1 | + | + | + |
| glucose-6- | + | + | − | H.2 | − | + | − |
| phosphatase | | | | | | | |
| catalase | − | + | − | H.4 | − | + | − |
| M2-PK | + | fetal | + | H-4 | ? | + | − |
| L-PK | − | + | fetal | H-6 | − | + | − |
| P450 mono- | + | + | − | HES₆ | − | + | − |
| oxygenase | | | | | | | |
| p-glycoprotein | ? | canaliculi | − | RL16/79 | − | postnatal | − |
| CK7 | − | − | + | RL23/36 | − | + | − |
| CK8 | + | + | + | BPC₅ | + | − | − |
| CK14 | + | − | − | Vimentin | − | − | fetal |
| CK18 | + | + | + | HepPar1 | + | + | − |
| CK19 | − (+) | − | + | Cell-CAM 105 | + | + | − |
| CKX | + | − | + | DPP IV | + | canaliculi | + |
| BDS₇ | + | − | + | lectin binding sites | + | − | + |
| OV1 | + | − | + | blood group | + | − | + |
| | | | | antigens | | | |
| OV6 | − | − | + | | | | |

Other hepatocyte markers are HNF-1 and transthyretin.

Cell markers specific for cardiomyocytes and their precursors include Cardiac troponin I (cTnI), Cardiac troponin T (cTnT), Nk×2.5, Atrial natriuretic factor (ANF), myosin heavy chain (MHC, particularly the β chain which is cardiac specific), Titin, tropomyosin, α-sarcomeric actinin, desmin, GATA-4, MEF-2A, MEF-2B, MEF-2C, MEF-2D, N-cadherin, Connexin 43, β1-adrenoceptor (β1-AR), creatine kinase MB (CK-MB), myoglobin, α-cardiac actin, atrial myosin light chain, ventricular myosin light chain, myocardin, myosin light chain 2v, and atrial natriuretic peptide.

Cell markers specific for neural lineage cells include A2B5 (a glycolipid) and polysialylated Neural Cell Adhesion Molecule (abbreviated NCAM), which can sometimes be displayed on other cell types, such as liver or muscle cells. Markers for neuronal cells include β-tubulin III, microtubule-associated protein 2 (MAP-2), and Nestin, characteristic of neural precursors and other cells. MAP-2 is a more stringent marker for fully differentiated neurons of various types. Other markers for neuronal cells include neurofilament heavy chain, neurofilament heavy chain, dopamine receptor d1, serotonin receptor 2a, serotonin receptor 5a, and dopa decarboxylase. Markers for oligodendrocyte cells present depending on the maturity of the cell population include NG2, galactocerebroside (GaIC), myelin basic protein (MBP), PDGFRα, a membrane receptor for PDGF, and TRα1.

Constitutive markers for other cell types can be chosen from the literature based on the known phenotype of the cells. Tissue specific promoters for use in this invention can be cloned directly from the corresponding gene, if the marker is a protein; or from an enzyme that catalyses synthesis of he marker, if the marker is a carbohydrate.

Reporter Genes

To detect potential up- or down-regulation by a promoter in response to metabolic or toxicologic change in the culture environment, it is operatively linked to a reporter gene that generates a detectable signal.

The reporter gene can encode a protein that produces a fluorescent or phosphorescent signal when expressed in the hepatocyte. In this way, behavior of the promoter sequence can be measured in situ. Autofluorescent proteins can be selected from humanized renilla green fluorescent protein (hrGFP), enhanced green fluorescent protein (eGFP), enhanced blue fluorescent protein (eBFP), enhanced cyan fluorescent protein (eCFP), enhanced yellow fluorescent protein (eYFP), or red fluorescent protein (RFP or DsRed). Bioluminescent proteins include firefly luciferase and Renilla luciferase. Enzymes that can be used to convert chemoluminescent substrates include alkaline phosphatase, peroxidase, chloramphenicol acetyl transferase, and β-galactosidase.

Also contemplated (for example, for use with automated systems) are reporters that generate a signal detectable by other means. Exemplary are genes which when expressed cause release of a biomolecule into the medium, or cause catalysis of a substrate in the medium into a detectable product. Regulation of the promoter can then be followed by assaying the biomolecule or the catalyzed product in the culture supernatant, for example, by immunoassay.

Producing the Genetically Altered Cells

Once a promoter-reporter system has been selected, the cells are genetically altered by standard recombinant techniques to place the reporter gene under control of the promoter. This can be done by transfecting the cells with a vector wherein the promoter and reporter are both heterologous to the cell, and already linked as an expression cassette. The cassette can then be placed into the genome in a random fashion. Alternatively, the user can place a heterologous reporter under control of an endogenous reporter by homologous recombination. This has the advantage of placing the promoter into a location in the genome known to be permissive for transcription under appropriate circumstances.

In principle, the genetic alteration can be done before or after the hES cells are differentiated—as long as there is sufficient replicative capacity in the transfected population to enable selection of the transfected cells. One of the advantages of working with hES cells is the ability to bulk up the population by any extent desired before differentiation. For this reason, the reader may prefer to undertake the genetic alteration while the hES cells are still in the undifferentiated state. In this way, they can subsequently generate an ongoing supply of undifferentiated and differentiated cells for further use. However, transfection and selection must be done under conditions selected to avoid premature differentiation of the cells.

Methods for genetically altering undifferentiated hES cells are described extensively in US 2002/0168766 A1. Plasmid vector systems such as Lipofectamine 2000™ (Gibco Life Technologies) or FuGENE™ (Roche Diagnostic Corporation), and viral vector systems based on retrovirus or lentivirus are all suitable. Selection of transfected hES cells can be done on antibiotic resistant feeder cells, or in a feeder-free culture environment (US 2002/0168766 A1).

As already indicated, there may be multiple promoter-reporter systems in the same cell line: for example, a first promoter that responds to a metabolic or toxicologic change in the culture environment linked to a first reporter, plus a second promoter that is tissue specific, linked to a second reporter. Alternatively or in addition, there may be one or more additional promoters that respond to the culture environment in a different fashion, representing another potential response to metabolic or toxicologic change. Again, the additional promoters are each linked to reporters that are distinguishable from the other reporters. For example, for use in fluorescent assays, transcription of each reporter will generate a gene or enzyme product that emits fluorescence at a different wavelength. Using this strategy, a cell population can report on different potential metabolic assaults at the same time.

Allelic Variants

Another benefit of using hES cells is the ability to make cells that are identical in all respects, except that they have a particular variation in the gene encoding a drug metabolizing enzyme or drug target of particular interest. This is relevant in the context of drug screening, because there are some naturally occurring allelic variants that affect an individual's ability to respond to or metabolize drugs of a particular class. Because the cells are otherwise the same, the user can determine the effect of the compound being screened in an allotype specific manner. See published U.S. patent application 2003/0003573 A1, paragraphs [0128] to [0134] (Geron Corp.).

Examples of drug metabolizing enzymes having known allelic variants of consequence are described by Wolf et al., Br. Med. J. 320:987, 2000; Wolf et al., Br. Med. Bull. 55:366, 1999; W. Webber, Pharmacogenetics. Oxford Univ. Press, 1997.

TABLE 2
Naturally Occurring Allotype Variants of Drug Metabolizing Enzymes

| | | | Total No. | |
|---|---|---|---|---|
| Enzyme | Variant phenotype | Frequency | of Drugs | Exemplary Substrates |
| CYP2D6 | poor metabolizer | White 6%; African | >100 | codeine, nortryptiline, |
| | | American 2%; Oriental 1% | | dextromethorphan |
| | ultra-rapid metabolizer | Ethiopian 20%; Spanish 7%; | | |
| | | Scandinavian 1.5% | | |
| CYP2C9 | reduced activity | | >60 | tolbutamide, diazepam, |
| | | | | ibuprophen, warfarin |
| CYP2C19 | poor metabolizer | Oriental 23%; White 4% | >50 | mephenytoin, omeprazole, |
| | | | | proguanil, citalopram |
| N-acetyl transferase | poor metabolizer | White 60%; African | >15 | isoniazid, procainamaide, |
| | | American 60%; Oriental 20%; | | sulphonamides, hydralazines |
| | | Inuit 5% | | |
| Thiopurine | poor metabolizer | low in all populations | <10 | 6-mercaptopurine, |
| methyltransferase | | | | 6-thioguanine, azathioprine |

To put into effect this embodiment of the invention, hES cells are divided into two or more separate cell populations. One or more of the cell population is genetically altered to introduce a variant of the gene for the drug metabolizing enzyme or drug target (before or after instruction of the promoter-reporter construct). The gene can be introduced by random transduction, but more typically the variant is substituted for the native gene by homologous recombination. This both silences the endogenous gene, and places the variant under control of endogenous transcription control elements. Alternatively, if a naturally occurring variant is known to differ from the usual gene by a point mutation, the endogenous gene can be mutated so as to confer the same phenotype. The user has the option of altering the opposite allele to express the same variant, or inactivating it, for example, by homologous recombination.

The cells are then differentiated and used for drug screening as described in the sections that follow.

Differentiating Cells to a Desired Tissue Type

Once the hES cells have been genetically altered with the promoter-reporter system(s) designed for expression in the test cell population, the population can be bulked up to any extent required, and then differentiated at will into the desired tissue type.

Liver Cells

Hepatocytes can be differentiated from hES cells using an inhibitor of histone deacetylase, as described in U.S. Pat. No. 6,458,589 and PCT publication WO 01/81549 (Geron Corporation). Undifferentiated hES cells are cultured in the presence of an inhibitor of histone deacetylase. In an exemplary method, differentiation is initiated with 1% DMSO, then with 2.5 mM of the histone deacetylase inhibitor n-butyrate. The cells obtained can be matured by culturing 4 days in a hepatocyte culture medium containing n-butyrate, DMSO, plus growth factors such as EGF, hepatocyte growth factor, and TGF-α.

Staged protocols for differentiating hES cells into hepatocytes are described in US 2005/0037493 A1 (Geron Corp.). Cells are cultured with several combinations of differentiation and maturation agents in sequence, causing the hES cells to differentiate first into early endoderm or hepatocyte precursors, and then to mature hepatocyte-like cells.

Differentiation into endoderm-like cells can be initiated using either butyrate, DMSO or fetal bovine serum, optionally in combination with fibroblast growth factors. Differentiation can then continue using a commercially available hepatocyte culture medium, including factors such as hepatocyte growth factor (HGF), epidermal growth factor (EGF), and/or bone morphogenic protein (e.g., BMP-2, 4, or 7) in various combinations. Final maturation may be enhanced by the presence of agents such as dexamethazone or Oncostatin M. An illustration of the “DMSO Protocol” from US 2005/0037493 A1, as applied to the reporter hepatocytes of this invention, is provided below in Example 3.

The term “hepatocyte” or “hepatocyte lineage cell” as used in this disclosure means a cell that has at least three, and preferably five or seven of the following characteristics: α₁-antitrypsin; asialoglycoprotein, glycogen storage, cytochrome P450 enzyme expression; glucose-6-phosphatase activity, low to negligible α-fetoprotein, and morphological features of hepatocytes (cuboidal cells, possibly with canalicular spaces between them). Assay methods for identifying markers are detailed in U.S. Pat. No. 6,458,589. Unless indicated or implied otherwise, a “hepatocyte” of this invention may or may not have been obtained by differentiating human embryonic stem cells.

In the context of drug screening, the user may also wish to test the activity of particular drug metabolizing enzymes, such as cytochrome P450 enzymes. A convenient way of surveying the activity of cytochrome P450 is to combine the cells with a “cassette” of substrates: such as midazolam (metabolized by CYP3A4), tolbutamide (metabolized by CYP2C9), phenacetin (CYP1A2), and bufuralol (CYP2D6). Activity can be quantitated as being about 0.1, 1, or 10 times that of a reference cell line, such as HepG2 cells. A convenient way of monitoring metabolites of all the drugs in the cassette simultaneously is by GCMS. If desirable, the cells can be treated with compounds such as dexamethazone or rifampicin before or during use in drug screening, so as to increase cytochrome P450 expression or activity in the cells.

Nerve Cells

Neural cells can be generated from hES cells according to the method described in U.S. Pat. No. 6,833,269; Carpenter et al., Exp Neurol. 2001; 172(2):383-97; and WO03/000868 (Geron Corporation). Undifferentiated hES cells or embryoid body cells are cultured in a medium containing one or more neurotrophins and one or more mitogens, generating a cell population in which at least ˜60% of the cells express A2B5, polysialylated NCAM, or Nestin and which is capable of at least 20 doublings in culture. Exemplary mitogens are EGF, basic FGF, PDGF, and IGF-1. Exemplary neurotrophins are NT-3 and BDNF. The use of TGF-β Superfamily Antagonists, or a combination of cAMP and ascorbic acid, can be used to increase the proportion of neuronal cells that are positive for tyrosine hydroxylase, a characteristic of dopaminergic neurons. The proliferating cells can then be caused to undergo terminal differentiation by culturing with neurotrophins in the absence of mitogen.

Oligodendrocytes can be generated from hES cells by culturing them as cell aggregates, suspended in a medium containing a mitogen such as FGF, and oligodendrocyte differentiation factors such as triiodothyronine, selenium, and retinoic acid. The cells are then plated onto a solid surface, the retinoic acid is withdrawn, and the population is expanded. Terminal differentiation can be effected by plating on poly-L-lysine, and removing all growth factors. Populations can be obtained in which over 80% of the cells are positive for oligodendrocyte markers NG2 proteoglycan, A2B5, and PDGFRa, and negative for the neuronal marker NeuN. See PCT publication WO 04/007696 and Keirstead et al., J. Neurosci. 2005; 25(19):4694-705. Derivation of retinal pigment epithelial cells has also been reported (Klimanskaya et al., Cloning Stem Cells 6:217, 2004).

Heart Cells

Cardiomyocytes or cardiomyocyte precursors can be generated from hES cells according to the method provided in WO 03/006950. The cells are cultured in suspension with fetal calf serum or serum replacement, and optionally a cardiotrophic factor that affects DNA-methylation, such as 5-azacytidine. Alternatively, cardiomyocyte clusters can be differentiated directly on a solid substrate using a combination of Activin A and Bone Morphogenic Protein 4. Spontaneously contracting cells can then be separated from other cells in the population, by density centrifugation.

Further process steps can include culturing the cells so as to form clusters known as cardiac bodies™, removing single cells, and then dispersing and reforming the cardiac bodies™ in successive iterations. Populations are obtained with a high proportion of cells staining positive for cTnI, cTnT, cardiac-specific myosin heavy chain (MHC), and the transcription factor Nk×2.5. See WO 03/006950, Xu et al., Circ Res. 2002; 91(6):501-8; and PCT/US2005/009081 (Geron Corporation).

Other Cell Types

Islet cells can be differentiated from hES cells by initiating differentiation of hES cells by culturing in a medium containing a combination of several factors selected from Activin A, a histone deacetylase inhibitor (such as butyrate), a mitogen (such as bFGF); and a TGF-β Superfamily antagonist (such as noggin). The cells can then be matured by culturing with nicotinamide, yielding a cell population in which at least 5% of the cells express Pdx1, insulin, glucagon, somatostatin, and pancreatic polypeptide. See WO 03/050249 (Geron Corp.).

Hematopoietic cells can be made by coculturing hES cells with murine bone marRow cells or yolk sac endothelial cells was used to generate cells with hematopoietic markers (U.S. Pat. No. 6,280,718). Hematopoietic cells can also be made by culturing hES cells with hematogenic cytokines and a bone morphogenic protein, as described in US 2003/0153082 A1 and WO 03/050251 (Robarts Institute).

Mesenchymal progenitors and fibroblasts can be generated from hES cells according to the method described in WO 03/004605. hES-derived mesenchymal cells can then be further differentiated into osteoblast lineage cells in a medium containing an osteogenic factor, such as bone morphogenic protein (particularly BMP-4), a ligand for a human TGF-β receptor, or a ligand for a human vitamin D receptor (WO 03/004605; Sotile et al., Cloning Stem Cells 2003; 5(2):149-55). US 2004/0009589 A1 (Iskovitz-Elder et al.) and US 2003/0166273 A1 (Kaufman et al., Wisconsin) report endothelial cells derived from human embryonic stem cells. Chondrocytes or their progenitors can be generated by culturing hES cells in microaggregates with effective combinations of differentiation factors listed in WO 03/050250 (Geron Corp.).

Other differentiation methods known in the art or subsequently developed can be used in conjunction with this invention to create promoter-reporter cells representative of other tissues.

Use of Reporter Cells for Drug Screening

Cells of this invention containing a promoter-reporter system can be used to screen for factors (such as solvents, small molecule drugs, peptides, and polynucleotides) or environmental conditions (such as culture conditions or manipulation) that affect metabolic properties or gene expression profiles. This can be done to study drug metabolism and drug safety, to identify factors that affect the regulation of drug metabolism, or to evaluate and validate a pharmacological effect. Of particular interest is the use of these cells in the screening of small molecule drugs, and other agents capable of entering the cell and/or altering cell metabolism in vivo, as part of pharmaceutical development.

Toxicity Testing

Use of the promoter-reporter cells of this invention in toxicity testing involves combining the cell population with the agent to be screened (typically by adding it to the medium). The effect of the agent on the promoter-reporter system is followed typically by comparing the signal from the reporter gene in the presence and absence of the agent, using a detection system appropriate for the reporter chosen.

By way of illustration, hES cells are genetically modified and differentiated to create a population of hepatocytes containing a promoter for heme oxygenase 1, linked to a green fluorescent protein reporter gene. Fluorescence of the population is measured, and then the cells are combined with the test agent in the same medium, and fluorescence is measured again. Increase in fluorescence level indicates that the heme oxygenase 1 gene is up-regulated, apparently in response to oxidative stress induced by the test agent. Different agents and agent combinations can be screened in a rapid throughput process, for example, by establishing the cells in the wells of a microtiter plate.

Depending on the differentiation protocol, cell populations can be used that are at least 50%, 80%, or 90% homogeneous for the cell type of interest. Where the cell populations are relatively pure, or when the selected promoter is only active in the cell type of interest (e.g., the CYP3A4 promoter in hepatocytes), then effects of the test agent on the target cell can be measured simply by following signal from the reporter gene in the cell population as a whole.

However, when the cell populations are more heterogeneous, and the promoter can be induced in more than one of the cell types present, then it may be preferable to follow the effect on a cell-by-cell basis. A cell that contains both a metabolic-responsive promoter-reporter system and a tissue-specific promoter-reporter system is equipped to do this particularly well. The test agent is combined with the cell population as a whole, but the output of the assay is measured as a change in the metabolic-responsive reporter when present in a cell labeled with the tissue specific reporter. A benefit of this approach is that there is no need for the target cell type to predominate the reagent cell population. Populations comprising less than 20%, 10%, or 5% of the target cells can be used, since a drug-induced effect will be demonstrated if there are detectable cells in which both reporters are expressed. This enables the drug screening techniques of this invention to be used with relatively rare cell types or subtypes—e.g., insulin-producing pancreatic islet cells, or neural cells that utilize a particular neurotransmitter.

Cell populations equipped with a plurality of metabolic or toxicologically responsive promoter-reporter systems (either as different promoter-reporter constructs in a single cell line, or in a population of mixed cells containing different promoter-reporter constructs) can be used to monitor multiple assault pathways simultaneously, as long as the products of the reporter genes are distinguishable.

Screening for Pharmacological Effect

Besides screening test compounds for toxicology, drug metabolism, and disposition, the cells of this invention can also be used to screen for positive pharmacological effect. For example, pancreatic cells containing a reporter system driven by an insulin promoter can be used to screen drugs capable of inducing insulin secretion. Neuronal cells containing a reporter system driven by promoters for genes in neurotransmitter synthesis, release, or uptake can be used to screen drugs with a potentially beneficial neurological effect. In tandem, matched populations of hepatocyte reporter cells can be used to screen for toxicological effects of the same compounds. In principle, the use of cells, kits, and methodology of this invention for positive screening parallels that of toxicity testing, selecting appropriate promoter systems and adapting the assays as appropriate.

Validation of Drug Targets and Drug Metabolizing Enzymes

During the course of screening for a toxicological or pharmaceutical effect, the user may wish to validate the presumed target of a particular drug, or an enzyme believed to be involved in its metabolism. This can be done by combining the drug with promoter-reporter cells in the presence or absence of a substance that either activates or inhibits transcription or translation of the drug target or metabolizing enzyme. The promoter-reporter construct is chosen to reflect gene activity downstream from the activity being tested. The user then determines whether there is a difference in expression of the reporter gene in the presence of the drug with or without the RNAi, as an indication of whether the drug does influence the drug target or enzyme in question.

Suitable inhibitors for use in this context are RNA molecules (RNAi) of the single or double stranded variety, having a sequence that enables it to inactivate translation in a gene specific manner. The synthesis and use of RNAi molecules and other inhibitors suitable for use in this context are well described in the art. See, for example, Huan et al., Cancer Res. 64:4294, 2004; Chan et al., Drug Discov. Today 10:587, 2005; M. Manoharan, Curr. Opin. Chem. Biol. 8:570, 2004; WO 04/094595; WO 05/014782). Other suitable activators and inhibitors include small molecule drugs known to upregulate or downregulate the gene at the transcription level (Campbell et al., J. Cell Sci. 109:2619, 1996).

Known drug targets include G protein-coupled receptors (GPCRs), activated by ligands like TNF; peroxisome proliferation-activated receptors (PPARs), which binds muraglitazar and other compounds; cytochrome P450 regulators like PXR, which are activated by dexamethazone, rifampicin, or pregnenalone 16α-carbonitrile; the nuclear receptor CAR, which are activated by phenobarbital and other barbiturates; Phase II enzymes like glycosyl transferase, which process polychlorinated biphenyl compounds; aryl hydrocarbon (Ah) receptors, which bind benzo[a]pyrene and β-naphthoflavone; and estrogen receptors, which bind estrogen analogs like tamoxifen.

Known drug metabolizing enzymes include the cytochrome P450 system (Ortiz de Montellano et al., supra), N-acetyl transferase, and enzymes involved in conjugation of bile acids and other compounds.

To illustrate this aspect of the invention, drug metabolism in the liver can be studied using hepatocytes having a promoter-reporter system that responds to oxidative stress. A drug that is metabolized through the cytochrome P450 system (e.g., phenobarbital) can be combined with the cells in the presence and absence of RNAi specific for particular P450 enzymes like CYP3A4. If there is higher reporter activity induced by the drug in the presence of the RNAi, then the reduction in CYP3A4 activity caused by the RNAi is resulting in increased stress—implicating CYP3A4 in the metabolic pathway of the drug.

In a similar fashion, role of the estrogen receptor in the pharmaceutical activity of a drug can be evaluated using cells having a promoter reporter system that reflects transcription of a gene up-regulated by estrogen. If there is lower reporter activity induced by the drug in the presence of RNAi specific for the estrogen receptor, then the estrogen receptor is validated as a target for the drug being tested.

Effect on Allelic Variants

Promoter-reporter cells made from the same hES cell line but engineered to contain different variants of a drug metabolizing enzyme can be used to compare the processing or effect of a drug thought to be metabolized by the enzyme. For example, hepatocytes derived from the same hES cell having the usual form of the CYP2D6 gene, can be compared with hepatocytes having the variant present in 6% of the population (Table 2) for the effect of a drug like dextromethorphan. Differences in drug metabolism attributable to the variation will affect the signal generated through a promoter-reporter that responds to metabolic or toxicologic changes in the cell, or reflects expression of a gene product implicated in metabolism of the drug.

In a similar fashion, promoter-reporter cells engineered to contain different variants of a drug target can be used to compare the effect of a drug on the target variants. For example, neuronal cells having variations in an enzyme involved in uptake of a neurotransmitter can be compared for the effect of a drug known to affect uptake (e.g., bupropion). Differences in the pharmacological effect of the drug attributable to the variation will affect the signal generated through a promoter-reporter that responds to presence of the neurotransmitter.

Separate cell populations having different variants of the drug target or drug metabolizing enzyme can be tested with the drug in parallel. Optionally, each variant can be placed in a cell population having different reporter genes. This enables the user to combine the two cell populations, and measure the effect of the drug on both variants together.

Use of Reporter Cells for Monitoring Transplanted Tissue

Another use of the promoter-reporter cells of this invention is to monitor the fate of a tissue graft (such as an allograft) transplanted into a human subject. Expression of the reporter can be used as an indicator of stress, apoptosis, or viability of the transplanted tissue, either because of inadequate engraftment, or because of subsequent acute or chronic immune rejection.

By way of example, a promoter chosen in this context could be one that responds to metabolic or toxicologic changes in the cell, such as a gene that responds to oxidative stress or apoptosis. A reporter is chosen that encodes a protein that is secreted by the cell, or causes secretion of another protein from the cell, permitting the effect to be monitored by sampling blood. Preferably, the reporter encodes a human protein or variant thereof, so as to minimize an immune response. Ideally, the reporter gene product is also excretable through the kidney, so that activity of the promoter can be monitored through the urine (WO 2004/090532, CXR et al.). Suitable candidates are human chorionic gonadotropin (hCG), major urinary protein, endostatin, β-lactoglobulin, and other hormones, proteins or peptides of relatively small size (<20,000, <10,000, or <5,000 kDa) to facilitate excretion and minimize kidney damage. Optionally, the reporter product can be a variant or fragment of the natural polypeptide, so as to be devoid of its normal biological activity. The promoter-reporter system can be transduced into the cell population as a transient genetic alteration (e.g., using a plasmid or adenovirus vector), so that the tissue ultimately becomes free of genetically altered cells some time after engraftment.

To use this aspect of the invention in the clinic, the promoter-reporter cells are combined with the tissue to be transplanted. Where the graft consists mainly of hES derived cells (e.g., hepatocytes, cardiomyocytes, or nerve cells), then the promoter-reporter cells can be mixed into the population at any time before transplantation. Where the graft consists mainly of tissue derived from other sources, such as a human donor, then the cells can be mixed together in the same fashion, or (for solid tissue grafts) the promoter-reporter cells can be implanted into the graft at multiple sites. The fate of the graft can then be monitored in situ at various intervals after transplant, by assaying blood or urine as appropriate.

Commercial Embodiments

As a commercial enterprise, the user may provide the screening methods of this invention as a service to other entities desiring to test the effect of their compounds or agents. Alternatively, the user may provide cells, combinations, and reagents of this invention to other entities for their internal use in pharmacologic screening.

The cells of this invention may be supplied in the form of a cell culture or suspension in an isotonic excipient or culture medium, optionally frozen to facilitate transportation or storage.

This invention also includes systems and kits useful for the derivation, production, or manufacture of hepatocytes having promoter-reporter system. The term “system” refers to a plurality of components designed for use in conjunction with each other, stored or used nearby one another, or under control of a single entity or partnership. Systems of this invention minimally comprise only the elements explicitly required, and may be present or operative in a larger structure or process designed for cell production or drug screening. The term “kit” refers to a plurality of components distributed or sold to another entity for use together in accordance with this invention. Optionally, a system or kit of this invention may contain one or more reagents useful for detecting the label (such as a lectin, antibody, or enzyme substrate), one or more control compounds known to induce or inhibit promoter activity (and thereby expression of the reporter), one or more RNAi molecules or other compounds that inhibit or otherwise influence a drug target or drug metabolizing enzyme in the cell population, and/or written information on the use of the cells and other components of the kit for drug screening or validation.

Products Comprising Multiple Cell Populations

Some systems or kits of this invention comprise two or more different cell populations maintained separately. The cell populations may be referred to as having the same genome. This means that genetic polymorphisms (RFLPs or SNPs) measured by standard techniques are consistent with them being derived from the same cell line (typically over 95% identical).

One such embodiment is a system or kit for producing differentiated promoter-reporter cells of this invention, comprising the genetically modified differentiated cells, and an hES cell line having the same genome (and possibly the same promoter-reporter construct) for making more of the differentiated cells as required.

Another such embodiment is a system or kit for drug screening, having a plurality of different cells sharing the same genome and the same promoter-reporter system, but differentiated into different cell lineages (e.g., hepatocytes and either cardiomyocytes or neural cells). This arrangement is particularly useful for testing the effect of drugs both for pharmacological effects on a target tissue (e.g., the heart or the central nervous system), and for toxic effects on the liver or other cell types.

Another such embodiment is a system or kit for drug screening, having a plurality of cells of the same differentiated cell type, sharing the same genome, but having different promoter-reporter systems so that different metabolic or toxicologic consequences of test compounds can be screened sequentially. Alternatively or in addition, different cell populations in the kit may be genetically identical except for introduced variants into a cell protein of interest, such as allelic variants of a cytochrome P450 enzyme. A promoter reflecting stress or toxicity is linked to the same or different reporter gene in each of the variants, so as to study the effect of compounds being screened as it relates to enzyme variants.

Other commercial applications will readily come to the mind of the skilled reader, and can be implemented within the scope of the claimed invention.

### The Following Examples Provided as Further Non-Limiting Illustrations of Particular Embodiments of the Invention

### EXAMPLES

### Example 1

### DMSO Protocol for Differentiating hES Cells

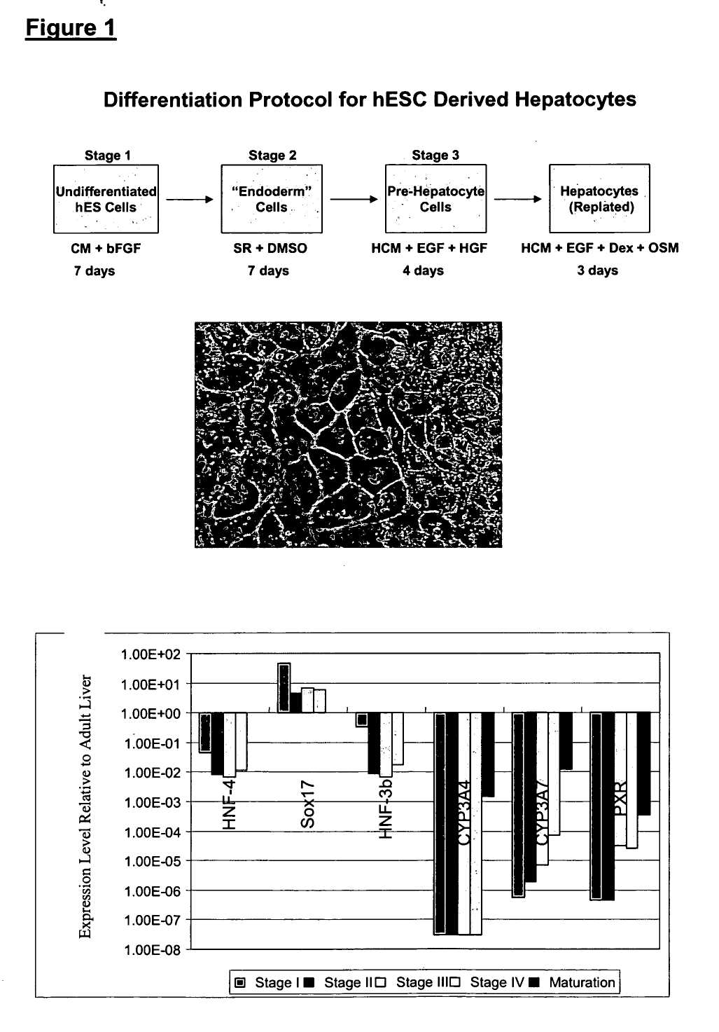

hES cells can be differentiated into hepatocytes according to the scheme shown in FIG. 1.

In one illustrative experiment, the human ES cells were plated at 1×10⁶ cells per 10 cm well, and grown in mEF conditioned medium containing 8 ng/mL added bFGF for 5 days, changing medium every day. Stage II/III was conducted by culturing the cells in KO-DMEM containing 20% Serum Replacement (Gibco # 10828-028), 2 mM L-glutamine, non-essential amino acids (NEAA), 0.1 mM β-mercaptoethanol, plus 1% DMSO. The medium was changed every day for 7 days.

Stage IV was then started by changing the medium to HCM containing 10 ng/mL EGF plus 2.5 ng/mL HGF. The medium was changed every day for 4 days.

The cells were then replated using trypsin or collagenase without scraping. Collagenase passaging was effected by removing supernatant, and adding 1 mL per well of 1 mg/mL Collagenase IV in KO-DMEM pre-warmed to 37° C. After a 5 min incubation, the collagenase was removed, and the cells were washed with PBS. 1 mL of medium was then added to the well, and the cells were then pipetted vigorously 20-30 times using a P1000 pipette. Under culture conditions where cells did not detach easily, trypsin/EDTA was used instead of collagenase. The washed cells were layered with 0.5 to 1 mL per well (Gibco # 25300-054, 0.05% trypsin, 0.53 mM EDTA), and incubated at 37° C. for 5 min. They were then dispersed by repeated pipetting, and the enzyme reaction was quenched with an equal volume of 10% FBS or soybean trypsin inhibitor. Large clumps were left behind, the cells were washed, and pelleted at 1200 rpm for 10 min. The cells were then suspended in new medium, and plated onto a 6 well plate.

The cells were then replated at ˜0.2 to 1×10⁶ cells per well, and grown for 15 days or until the wells looked confluent, changing the medium every 2-3 days. They were matured by culturing in the same medium containing 1 μM dexamethazone, plus either 10 ng/mL HGF or 10 ng/mL EGF, changing the medium every 2-3 days. The middle panel of FIG. 1 shows the cells after ˜15 days, demonstrating morphology characteristic of hepatocytes.

The lower panel of FIG. 1 shows analysis of expression of hepatocyte lineage markers, detected by real-time PCR, and normalized to the level expressed by samples of human adult liver. As cells pass through the maturation steps, the level of mRNA in the culture for cytochrome P450 enzymes CYP3A4, CYP3A7, and the P450 regulator PXR rise to a level that is closer to intact liver. Activity of CYP3A4 measured in an enzyme assay was activated by rifampicin and inhibited by ketoconozole, which is typical of CYP3A4 activity present in hepatocytes isolated from liver tissue.

### Example 2

### Generation of hESC Reporter Lines

The lab work for this example and the two examples that follow was conducted by Wei Cui, Debiao Zhao, David Hay, and Arlene Ross, at the Dept. of Gene Function & Development, Roslin Institute, Midlothian Scotland, for which the owners of the claimed invention wish to express their gratitude.

As a model for the reporter hepatocytes of this invention, the human α-fetoprotein and albumin promoters were amplified from human genomic DNA (Promega G3041) with specific primers listed in Table 2.

TABLE 3
Primers for Amplifying Hepatocyte Specific Promoters

| DNA | | DNA | Accession |
|---|---|---|---|
| fragment | Primer sequences (5′-3′) | size | No. |
| α-fetoprotein | Forward: (SEQ. ID NO:1) | 5.4 kb | NT006216 |
| | TTGTCGACTTGGGGACTATCTGATCTGGGG | | (330690- |
| | Reverse: (SEQ. ID NO:2) | | 336082) |
| | TTGGATCCGCCAGGGACTTCATGGTTGCTAG | | |
| albumin | Forward: (SEQ. ID NO:3) | 7.1 kb | NT006216 |
| | GACCCTGTTTTGACTAGTGGCTAG | | (2769969- |
| | Reverse: (SEQ. ID NO:4) | | 2777069) |
| | TAGGATCCATGGTTACCCACTTCATTGTGCC | | |

The lentiviral vector used for transfection of the hESCs was a modified version of the pLenti6N5-D-TOPO vector (Invitrogen). The CMV promoter fragment was removed, and a linker sequence containing multiple cloning site was inserted. The reporting plasmid cassettes were cloned into the modified lentiviral vector, and the lentiviruses were packaged according to the protocol provided by Invitrogen and tittered using HT1080 cells.

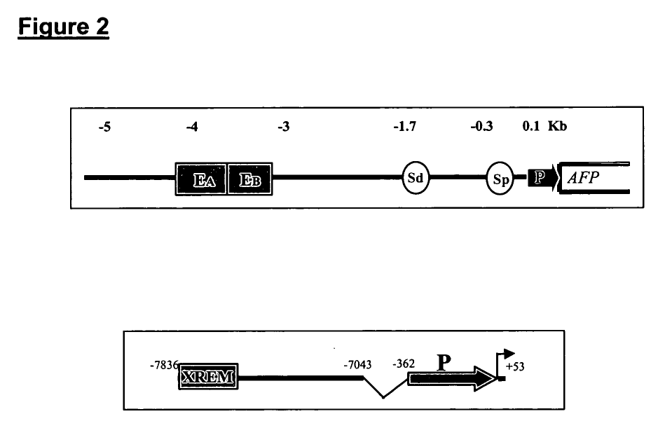

The fidelity of all the DNA sequences was confirmed by sequencing and restriction enzymes digestion. The correct promoter fragments were subsequently cloned into vector peGFP1 (Invitrogen) upstream of eGFP. The AFP promoter is 5.4 kb in size containing enhancer domains A and B and both proximal and distal silencers (FIG. 2A).

The human embryonic stem cell (hESC) line H1 was cultured in Matrigel®) coated plates using mouse embryonic fibroblast conditioned medium (mEF-CM) [Gerrard et al., infra]. Confluent H1 cells were split 1:3 by 0.5 mM EDTA treatment 24 h before transfection/transduction. The cells were transfected using either lipofectamine™ or Fugene6™ with a linearized plasmid as described previously [Xu et al., infra] or incubated with 10²⁻³TU/mL lentiviruses in CM containing bFGF (8 ng/mL) and polybrene (6 μg/mL) overnight. Selection was applied 48 h post-transfection/post-transduction, using G418 or blasticidin for 2-3 weeks. Surviving colonies were picked and expanded.

The phAFP-eGFP and phALB-eGFP were transiently transfected into hepatocarcinoma cell lines (HepG2 and Hep3B) as well as undifferentiated hESCs. The GFP expression was visible in transfected HepG2 and Hep3B cells but not in the hESCs, indicating that both promoters were specific for hepatocytes-like cells.

Lentiviral vectors carrying either hAFP-eGFP or hALB-eGFP cassette were then constructed by inserting the individual cassette into modified lentiviral vector, pLenti Z (FIG. 2C). The lentiviruses produced were used to transduce H1 hESCs. After selection with blasticidin, nine and fourteen clones were established for pLZ-hAFP-eGFP and pLZ-hALB-eGFP, respectively. Transgene integration has been confirmed by PCR amplification of the fragment crossing the promoter and eGFP joint region. The established transgenic cell lines are named H1-LZ-hAFP-eGFP and H1-LZ-hALB-eGFP respectively.

### REFERENCES

1. Watanabe K, Saito A, Tamaoki T., (1987) Cell-specific enhancer activity in a far upstream region of the human alpha-fetoprotein gene. J Biol Chem. 1987 262:4812-8.
2. Hayashi Y, Chan J, Nakabayashi H, Hashimoto T, Tamaoki T., (1992) Identification and characterization of two enhancers of the human albumin gene. J Biol Chem. 267:14580-5.
3. Xu C., Inokuma M. S., Denham J., Golds K., Kundu P., Gold J. D., Carpenter M. K. (2001) Feeder-free growth of undifferentiated human embryonic stem cells. Nat Biotechnol 19:971-4.
4. Gerrard L, Zhao D B, Clark A J, and Cui W (2005) Stably transfected human embryonic stem cell clones express OCT4-specific green fluorescent protein and maintain self-renewal and pluripotency. Stem Cells 23:124-33.

### Example 3

### Differentiation into Hepatocytes and Expression of Reporter Genes

Undifferentiated hESCs containing the reporter construct were cultured in mEF-CM containing 8 ng/mL bFGF until approximately 70% confluent. The CM was then replaced with SR/DMSO media (Knockout-DMEM containing 20% Serum Replacement, 2 mM I-Glutamine, 1×non-essential amino acids, 0.1 mM β-mercaptoethanol and 1% DMSO) and changed daily for 7 days. The cells were then matured using hepatocyte culture medium (HCM from Cambrex) supplemented with hepatocyte growth factor (HGF) 2.5 ng/mL and EGF 10 ng/mL for another 2 weeks. HCM was changed daily for the first 4 days and every second for the last 10 days. Cells were immunostained either directly following a PBS wash (cell surface antigen) [Gerrard et al., supra] or after fixation with 100% methanol.

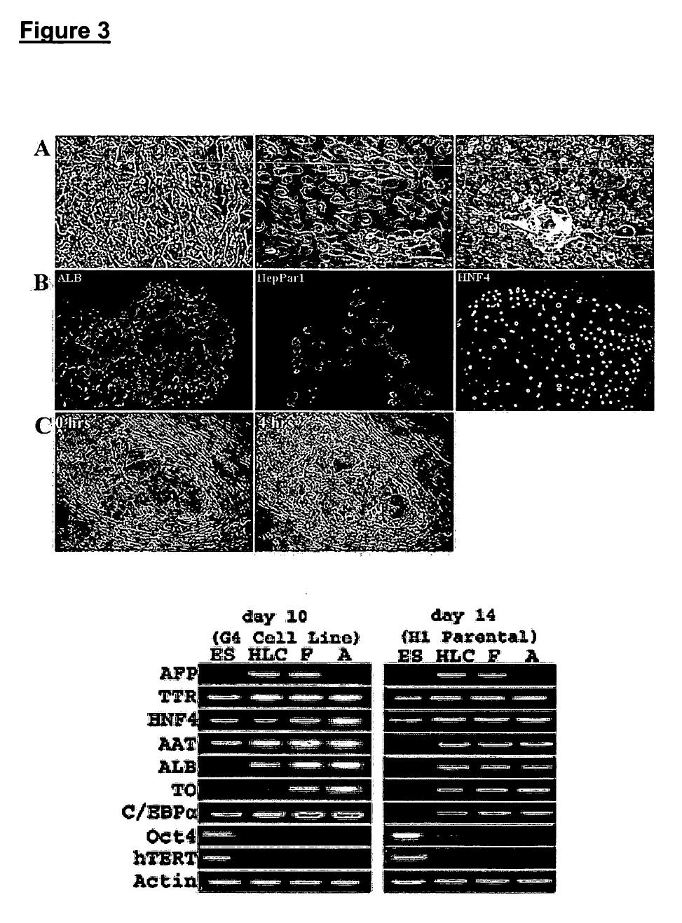

FIG. 3 shows the results. After DMSO treatment, cells exhibited endoderm-like morphology and following further culture in HCM (supplemented with HGF and EGF), hepatocyte lineage cells (HLCs) formed as foci that were polygonal in shape (FIG. 3A). This morphology was similar to that of primary human hepatocytes. The HLCs contained slightly bigger nuclei with prominent nucleoli, and were surrounded by structures that resembled bile canaliculi. They expressed hepatocyte markers, such as HNF4, albumin and HepPar1 (FIG. 3B).

To test the response of HLCs to toxins, the cells were grown in HCM containing Rifampicin (50 μM) and dexamethasone (1 μM) for 6 days, incubated in HCM medium containing 1 mg/mL indocyanine green (ICG) for 45 min, and then continuously cultured in HCM supplemented with HGF and EGF. The clearance of ICG was monitored every hour post-ICG treatment. The cells were capable uptake and clearance of ICG, which is characteristic of hepatocytes (FIG. 3C). HLCs comprised 10% to 30% of the total cell population. Within HLC foci, individual HLCs were at different stages of maturity, as determined by AFP immunostaining. RT-PCR studies using foci specific RNA showed that cells expressed a range of hepatocyte markers (FIG. 3D).

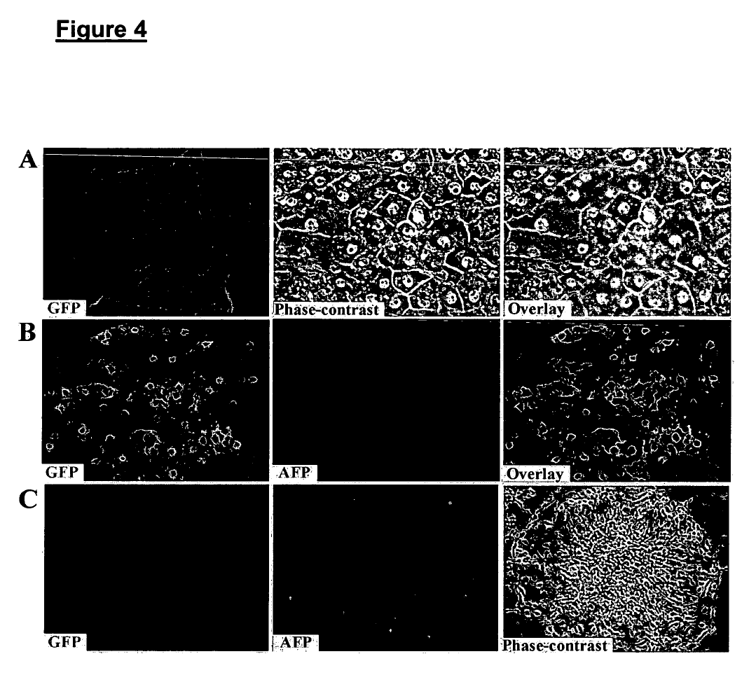

Eight H1-LZ-hAFP-eGFP clonal lines showed eGFP expression during later stages of hepatocyte differentiation (FIG. 4A). The GFP expression appeared exclusively in HLCs foci with 5-15% of the HLCs within each foci expressing GFP. To confirm that the GFP detected was associated with endogenous AFP expression, the cells were costained with both AFP and GFP antibodies, showing co-localization of AFP staining with GFP staining (FIG. 4B).

To determine whether hAFP-eGFP is also expressed in hESC-derived cells representing primitive endoderm, the H1-LZ-hAFP-eGFP cells were differentiated to form embryoid bodies (EB) in suspension, which were then plated out in culture. Double staining with anti-GFP and anti-AFP showed that only endogenous AFP expression was detected in primitive endoderm and not GFP expression (FIG. 4C). These results show that the 5.4 kBase hAFP upstream regulatory region we cloned is specific for cells committed to the hepatocyte cell lineage.

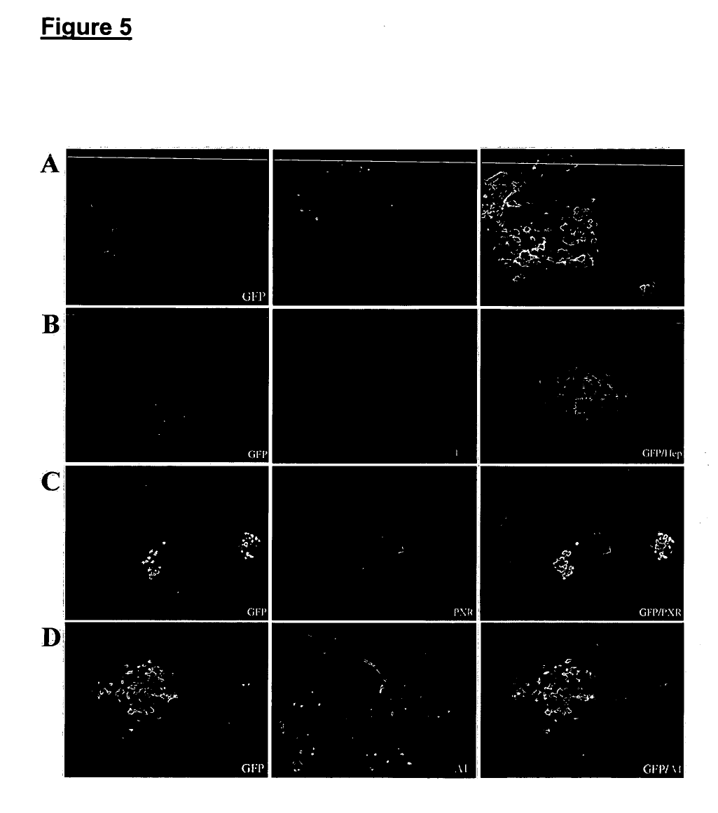

H1-LZ-hAFP-eGFP derived HLCs were stained with anti-GFP and anti-albumin antibodies and exhibited heterogeneous staining pattern for GFP and albumin (FIG. 5A). In some cells (possibly early stage hepatocytes), only hAFP-GFP expressed, whilst in other cells (possibly later stage hepatocytes), hAFP-GFP and albumin were either co-localized or only albumin was detected. Double staining showed that hAFP-GFP was co-localized with HepPar1, a marker for hepatoblasts (FIG. 5B) whilst hAFP-GFP did not show any co-localization with PXR or aAT, markers for mature hepatocytes (FIGS. 5C & D).

### Example 4

### Reporter Hepatocytes Driven by the CYP3A4 Promoter

The example described here produced hepatocytes having a GFP reporter driven by promoter elements for the human CYP3A4 gene from Cytochrome P450. The regulatory sequences contain two elements: the 0.8 kBase xenobiotic-responsive enhancer module (XREM) and the 0.4 kBase mini proximal promoter.

CYP3A4 promoters were cloned from human genomic DNA by PCR as in Example 2, using the primers shown in Table 3, confirmed by sequencing, and then linked together (FIG. 4B).

TABLE 4

Primers for Amplifying Hepatocyte Specific
Promoters

| DNA | | | |
|---|---|---|---|
| frag- | | DNA | Accession |
| ment | Primer sequences (5′-3′) | size | No. |
| XREM | Forward: (SEQ. ID NO:5) | 0.8 kb | AF195589.1 |
| of | GCTCTAGAGAGATGGTTCATTCC | | (2633-3425) |
| CYP3A4 | Reverse: (SEQ. ID NO:6) | | |
| | GATGGCATGCCAGTCTCATTGAT | | |
| Mini | Forward: (SEQ. ID NO:7) | 0.4 kb | AF195589.1 |
| pro- | AGGTAAAGATCTGTAGGTGTG | | (10101-10521) |
| moter | Reverse: (SEQ. ID NO:8) | | |
| of | TGTTGCTCTTTGCTGGGCTAT | | |
| CYP3A4 | | | |

The CYP3A4-eGFP construct was validated by transiently transfecting it into hESCs and HepG2 cells. eGFP expression was seen in HepG2 cells, but not hESCs, in response to rifampicin (50 mM) and dexamethasone (1 mM) treatment. The CYP3A4-eGFP construct was transfected into H1 cells by lipofectamine and 4 clones were shown to have correct transgene integrations by Southern blotting analysis. The CYP3A4-GFP hESCs were then differentiated into HLCs as in Example 2.

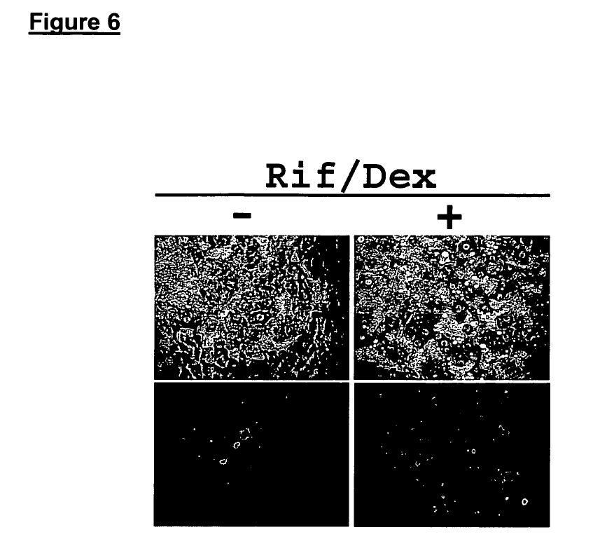

As shown in FIG. 6, two of the four CYP3A4-GFP hESC clonal lines showed detectable GFP expression upon chemical induction. The resulting HLCs were induced with both Rifampicin and dexamethasone for 6 days resulting in GFP expression monitored by fluorescent microscopy.

### REFERENCES

1. Goodwin B, Hodgson E, and Liddle C., (1999) The Orphan human pregnane×receptor mediates the transcriptional activation of CYP3A4 by rifampicin through a distal enhancer module. Mol Pharmacol. 56(6):1329-39.

### Example 5

### Cytochrome P450 Enzyme Activity in hESC Derived Reporter Hepatocytes

The lab work for this example was conducted by Cliff Elcombe and his team at CXR Biosciences, for which the owners of the claimed invention wish to express their gratitude.

To test enzyme activity in reporter hepatocytes engineered as described in Example 3, cells were contacted with a combination of P450 substrates, and monitored for drug metabolism by LC-MS/MS.

hESC derived hepatocytes containing the CYP3A4 promoter GFP reporter construct were compared with the HepG2 cell line (human hepatocyte carcinoma cells; ECACC No. 85011430) plated onto Matrigel®. The hES derived cells were maintained in Hepatocyte Basal Medium (Clonetics CC-3199) with the addition of HCM SingleQuots™ supplement (Clonetics CC-4182) omitting the EGF and Gentamycin. Penicillin and streptomycin were added to give final concentrations of 100 U/mL and 100 μg/mL respectively. The HepG2 cells were maintained in Dulbecco's Minimum Essential Medium with 10% Fetal Bovine Serum, 2 mM L-glutamine, 1% non-essential amino acids, 100 U/mL penicillin and 100 μg/mL streptomycin.

The cells were activated with 40 μM rifampicin with or without 1 μM dexamethasone, or with solvent control (DMSO) for 6 days. They were then combined with the following substrates:

To induce CYP3A4 (and possibly CYP2C9 & CYP2D6), the cells were treated with 40 μM rifampicin with or without 1 μM dexamethasone, with solvent) control (DMSO) for 6 days, or left untreated. The cells were then incubated with the following substrates in the absence of DMSO, rifampicin or dexamethasone for 24 h:

TABLE 5
Substrates for testing P450 Activity in hESC Derived Hepatocytes

| Substrate | | | |
|---|---|---|---|
| (final concentration) | | P450 isozyme | Metabolite produced |
| 50 | μM phenacetin | CYP1A2 | acetaminophen |
| 500 | μM tolbutamide | CYP2C9 | 4′-hydroxytolbutamide |
| 50 | μM bufuralol | CYP2D6 | 1′-hydroxybufuralol |
| 50 | μM midazolam | CYP3A4 | 1′-hydroxymidazolam |
| 50 | μM omeprazole | CYP2C19 | 5′hydroxyomeprazole |

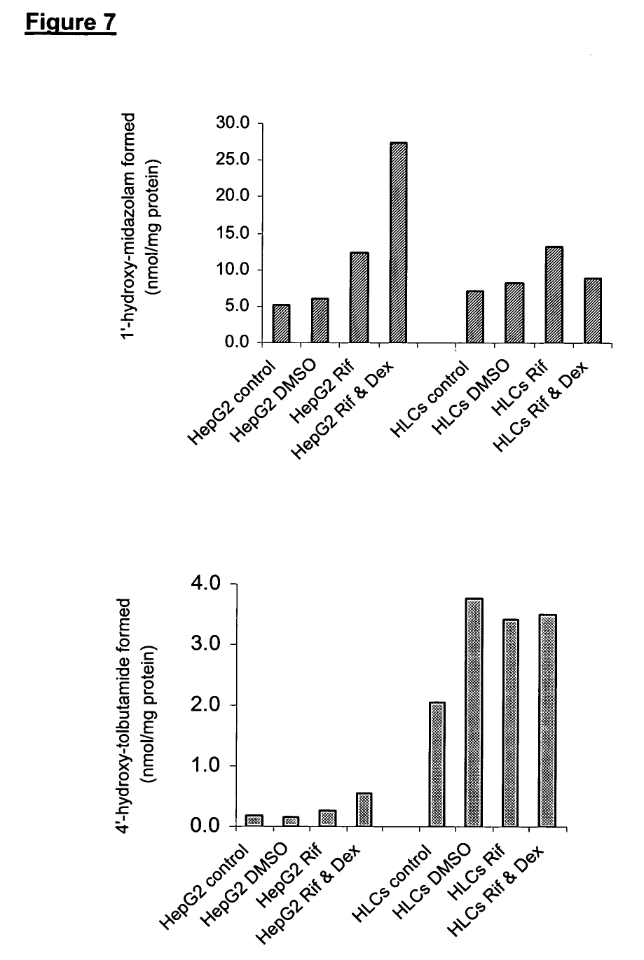

Metabolism of midazolam and tolbutamide by the HepG2 control cell line, and by the promoter-reporter HLCs is shown in FIG. 7. The data are adjusted for proportion of cells having the morphological characteristics of hepatocytes.

These results demonstrate that the hESC derived cells have inducible cytochrome P450 activity characteristic of pharmacologically active hepatocytes.

Other Patent Disclosures

Besides the textbooks and other references listed in the disclosure, the skilled reader may wish to consult the following patent disclosures:
hES cell culture: U.S. Pat. No. 6,800,480; WO 01/51616; WO 03/020920
Hepatocytes: U.S. Pat. No. 6,458,589; WO 01/81549; US 2003/0003573 A1; US 2005/0037493 A1
Neural cells: U.S. Pat. No. 6,833,269; WO 03/000868; WO 04/007696
Cardiomyocytes: WO 03/006950; PCT/US2005/009081
Islet cells: WO 03/050249
Other differentiated cells: WO 03/004605; WO 03/050250; WO 03/050251; US 2004/0009589 A1; and US 2003/0166273 A1.

As provided by U.S. law, the aforelisted patents and patent applications are hereby incorporated herein by reference in their entirety with respect to supporting information related to the features, culturing, and use of undifferentiated stem cells and cells differentiated therefrom that can be used in the context of the invention described here.

It is understood that certain adaptations of the invention are a matter of routine optimization, and can be implemented without departing from the claimed invention and its equivalents.

The invention claimed is:

1. A method of drug testing, comprising:
a) combining the drug with a cell population differentiated from human embryonic stem (hES) cells, comprising cells that have been genetically altered so that a promoter that responds to a metabolic or toxicologic change in the cell controls expression of a reporter gene; and then
b) determining whether there is a change in expression of the reporter gene.

2. The method of claim 1, wherein the cell population is a population of neural, cardiomyocyte, or hepatocyte lineage cells.

3. The method of claim 1, wherein the promoter responds to apoptosis, DNA damage, hyperplasia, oxidative stress, or a metabolic or toxicologic change in the cell.

4. The method of claim 1, wherein the promoter is a promoter for a gene selected from PUMA, p21, p21WAF1, Ki-67, heme oxygenase 1, superoxide dismutase, γ-glutamyl cysteinyl ligase, metallothionine, PXR, CAR, aryl hydrocarbon receptor (AhR), Nrf2 gene, prostate specific antigen, MDR1, CYP3A4, and CYP1A1.

5. The method of claim 1, wherein the cell population has also been genetically altered so that a second promoter that is tissue specific controls expression of a second reporter gene.

6. The method of claim 5, wherein the second promoter is a promoter for a hepatocyte specific marker selected from albumin, α1-antitrypsin, α-fetoprotein, γ-glutamyl tranpeptidase, glucose-6-phosphatase, catalase, and monooxygenase.

7. The method of claim 5, comprising determining whether there is a change in expression of the first reporter gene in cells expressing the second reporter gene.

8. The method of claim 1, wherein the cells have been genetically altered to express a gene encoding a variant of an endogenous drug target or drug metabolizing enzyme.

9. The method of claim 1, wherein the cell population has been obtained by:
i) genetically altering cells from said hES cell line, thereby producing hES cells in which a promoter that responds to a metabolic or toxicologic change in the cell controls expression of a reporter gene;
ii) proliferating the genetically altered hES cells to form a genetically altered hES cell population; and then
iii) differentiating the genetically altered hES cell population into a population of genetically altered differentiated cells.

10. The method of claim 1, comprising combining the drug with the cell population in the presence or absence of an RNAi for a drug target or drug metabolizing enzyme, and determining whether there is a difference in expression of the reporter gene in the presence of the drug with or without the RNAi.

11. A system or kit for drug testing according to the method of claim 1, comprising a cell population differentiated from human embryonic stem (hES) cells, in which at least some of the cells have been genetically altered so that a promoter that responds to a metabolic or toxicologic change in the cell controls expression of a reporter gene.

12. The system or kit of claim 11, further comprising a compound known to change expression of the reporter gene in the cells.

13. The system or kit of claim 11, comprising a plurality of cell populations sharing the same genome, but genetically altered such that different promoters that respond to a metabolic or toxicologic change control expression of a reporter gene in each cell population.

14. The system or kit of claim 11, comprising a plurality of cell populations sharing the same genome, but differentiated into cells of different tissues.

15. The system or kit of claim 11, comprising a plurality of promoter-reporter cell populations sharing the same genome, but having different variants of said drug target or drug metabolizing enzyme.

16. The system or kit of claim 11, wherein the variants are allelic variants of CYP2D6, CYP2C9, CYP2C1 g, N-acetyl transferase, or thiopurine methyltransferase.

17. Isolated tissue prepared for transplantation and adapted for monitoring after engraftment into a subject, containing cells differentiated from hES cells that have been genetically altered so that a promoter that responds to a metabolic or toxicologic change in the cell controls expression of a reporter gene.

18. The isolated tissue of claim 17, wherein the reporter gene in the promoter-reporter cell population encodes an excretable reporter.

19. The isolated tissue of claim 18, wherein the excretable reporter is hCG.

20. A method for evaluating a tissue allograft in a subject, comprising grafting the subject with tissue according to claim 17, and monitoring the tissue after engraftment by expression of the reporter gene.


SEQUENCE LISTING (8 sequences)

SEQ ID NO: 1 - DNA, 30 bp, Homo sapiens
ttgtcgacttggggactatctgatctgggg

SEQ ID NO: 2 - DNA, 31 bp, Homo sapiens
ttggatccgccacccacttcatggttgctag

SEQ ID NO: 3 - DNA, 24 bp, Homo sapiens
gaccctgttttgactagtggctag

SEQ ID NO: 4 - DNA, 31 bp, Homo sapiens
taggatccatggttacccacttcattgtgcc

SEQ ID NO: 5 - DNA, 23 bp, Homo sapiens
gctctagagagatggttcattcc

SEQ ID NO: 6 - DNA, 23 bp, Homo sapiens
gatggcatgccagtctcattgat

SEQ ID NO: 7 - DNA, 21 bp, Homo sapiens
aggtaaagatctgtaggtgtg

SEQ ID NO: 8 - DNA, 21 bp, Homo sapiens
tgttgctctttgctgggctat